(19) United States
(12) Patent Application Publication
(10) Pub. No.: US 2017/0009277 A1
(43) Pub. Date: Jan. 12, 2017

(54) GENETIC RESISTANCE TESTING

(71) Applicant: SIEMENS HEALTHCARE GMBH, Erlangen (DE)

(72) Inventors: Christina Backes, Saarbrücken (DE); Andreas Keller, Püttlingen (DE); Jan Kirsten, Bamberg (DE); Gabriel Rensen, Ukiah, CA (US); Susanne Schmolke, Erlangen (DE); Cord Friedrich Stähler, Hirschberg an der Bergstraße (DE)

(21) Appl. No.: 15/115,322

(22) PCT Filed: Jan. 30, 2015
(86) PCT No.: PCT/EP2015/051926

§ 371 Date: Jul. 29, 2016

(30) Foreign Application Priority Data
Jan. 30, 2014 (EP) 14153260.6
Aug. 1, 2014 (EP) EP14179456

(51) Int. Cl. C12Q 1/68

(52) U.S. Cl. CPC C12Q 1/689; C12Q 2600/156

(57) ABSTRACT

The embodiments relate to a method of determining an antibiotic resistance profile for a bacterial microorganism and to a method of determining the resistance of a bacterial microorganism to an antibiotic drug, wherein the bacterial microorganism belongs to the species Escherichia coli (E. coli). The method includes determining a nucleic acid sequence information or determining the presence of a mutation of at least one gene.

The present patent document is a §371 nationalization of PCT Application Serial Number PCT/EP2015/051926, filed Jan. 30, 2015, designating the United States, which is hereby incorporated by reference, and this patent document also claims the benefit of EP 14153260.6, filed Jan. 30, 2014, and EP 14179456.0, filed Aug. 1, 2014, which are also hereby incorporated by reference.

### TECHNICAL FIELD

The embodiments relate to a method of determining an antibiotic resistance profile for a bacterial microorganism and to a method of determining the resistance of a bacterial microorganism to an antibiotic drug.

### BACKGROUND

Antibiotic resistance is a form of drug resistance whereby a sub-population of a microorganism, (e.g., a strain of a bacterial species), may survive and multiply despite exposure to an antibiotic drug. It is a serious and health concern for the individual patient as well as a major public health issue. Timely treatment of a bacterial infection requires the analysis of clinical isolates obtained from patients with regard to antibiotic resistance, in order to select an efficacious therapy.

Antibacterial drug resistance (ADR) represents a major health burden. According to the World Health Organization's antimicrobial resistance global report on surveillance, ADR leads to 25,000 deaths per year in Europe and 23,000 deaths per year in the US. In Europe, 2.5 million extra hospital days lead to societal cost of 1.5 billion euro. In the US, the direct cost of 2 million illnesses leads to 20 billion dollar direct cost. The overall cost is estimated to be substantially higher, reducing the gross domestic product (GDP) by up to 1.6%.

Currently, resistance/susceptibility testing is carried out by obtaining a culture of the suspicious bacteria, subjecting it to different antibiotic drug protocols and determining in which cases bacteria do not grow in the presence of a certain substance. In this case, the bacteria are not resistant (e.g., susceptible to the antibiotic drug) and the therapy may be administered to the respective patients. U.S. Pat. No. 7,335,485 describes a method of determining the antibiotic susceptibility of a microorganism, wherein the organism is cultured in the presence of an antibiotic drug to be tested. More recently, sensitive technologies as Mass Spectrometry are applied to determine resistance, but this still requires culturing of the microorganism to be tested in the presence of an antibiotic drug to be tested. Further, in all these techniques, each microorganism to be tested has to be tested against individual antibiotic drugs or drug combinations, requiring extensive, time-consuming, and cumbersome tests.

It is known that drug resistance may be associated with genetic polymorphisms. This holds for viruses, where resistance testing is established clinical practice (e.g., HIV genotyping). More recently, it has been shown that resistance has also genetic causes in bacteria and even higher organisms, such as humans where tumors resistance against certain cytostatic agents may be linked to genomic mutations.

Wozniak et al. (BMC Genomics 2012, 13(Suppl 7):523) disclose genetic determinants of drug resistance in Staphylococcus aureus based on genotype and phenotype data. Stoesser et al. disclose prediction of antimicrobial susceptibilities for Escherichia coli and Klebsiella pneumoniae isolates using whole genomic sequence data (J Antimicrob Chemother 2013; 68: 2234-2244).

Escherichia coli (E. coli) is a Gram-negative, facultative anaerobic, rod-shaped bacterium potentially found, e.g., in the lower gastro-intestinal tract of mammals. While many species of the Escherichia genus are harmless, some strains of some species are pathogenic in humans causing urinary tract infections, gastrointestinal disease, as well as a wide range of other pathologic conditions. E. coli is responsible for the majority of these pathologic conditions.

### BRIEF SUMMARY AND DESCRIPTION

There remains a need for quick and efficient antibiotic resistance testing.

The scope of the present invention is defined solely by the appended claims and is not affected to any degree by the statements within this summary. The present embodiments may obviate one or more of the drawbacks or limitations in the related art.

Extensive studies were performed on the genome of E. coli bacteria resistant to antibiotic drugs and found remarkable differences to wild type E. coli. Based on this information, it is now possible to provide a detailed analysis on the resistance pattern of E. coli strains based on individual genes or mutations on a nucleotide level. This analysis involves the identification of a resistance against individual antibiotic drugs as well as clusters of them. This allows not only for the determination of a resistance to a single antibiotic drug, but also to groups of antibiotics such as lactam or quinolone antibiotics, or even to all relevant antibiotic drugs.

Therefore, the present embodiments will considerably facilitate the selection of an appropriate antibiotic drug for the treatment of an E. coli infection in a patient and thus will largely improve the quality of diagnosis and treatment.

According to a first aspect, the present embodiments are directed to a method of determining an antibiotic resistance profile for a bacterial microorganism belonging to the species E. coli including the acts of: a) providing a sample containing or suspected of containing the bacterial microorganism; b) determining the presence of a mutation in at least one gene of the bacterial microorganism selected from the group of genes listed in Table 4; wherein the presence of a mutation is indicative of a resistance to an antibiotic drug.

Table 4 is depicted in the following:

TABLE 4

abgB
allA
argI
caiC
csiE
cynX
dadX
elaD
fcl
fhuA
flhA
flu
frwC
gyrA
hofB
htrL
hybA
hyfB
hyfG
hyfI
hypF
ilvY
lsrC
lsrF
menE
mhpA
mnmC
mukB
norW
ompA
ompC
parC
pgaA
potB
puuC
puuE
rem
rhsC
rhsD
Rz
stfR
tilS
valS
xseA
yacH
yafE
yafT
yagR
yaiO
ybbB
ybfD
ybfQ
ycbF
ycbS
yccE
yceH
ycgB
ycgK
yciQ
ydbD
yddV
ydeK
ydjO
yeaU
yeaX
yeeJ
yefM
yegE
yegI
yehB
yehI
yehM
yeiI
yfaL
yfaW
yfcO
yfdF
yfdR
yfdX
yfhM
ygbN
ygcQ
ygeK
ygeO
ygiD
yhaC
yhaI
yhdP
yhgE
yhiJ
yjbI
yjcF
yjfF
yjfZ
yjgL
yjgN
yjhS
yjjJ
ymdC
ynbB
yncG
yneK
yphG
zraS
agaD
astE
chbG
eutE
eutQ
flgL
gcvP
gspO
gudD
hemF
kdpE
ldrA
livG
murB
murP
nepI
pphB
ptrB
rhaD
speC
tiaE
torZ
uidB
ycjX
ydiU
yejA
yfbL
yfiK
ygcR
ygcU
ygfZ
ygiF
ygjM
yjjU
yjjW
yohG
ypdB
yqjA
yrfB
ytfG
aspS
birA
cysD
dapB
dxs
eutA
fadA
fdx
fhuB
fhuC
fhuD
fmt
gudP
helD
hrpB
ilvA
kdpD
ldcA
lplA
menB
metH
pbpC
purH
purK
purL
queF
rhaA
rhaB
rplO
srlD
thiC
thiE
thiM
trpC
udp
uxaA
ybiB
ybiU
ydfI
ydgA
yecA
yehT
yfcN
yheN
yhgF
yhhQ
yhjE
YijG
ynfA

The presence or absence of a mutation in these genes is tested in relation to the reference strain E. coli K12 substrain DH10B (see also more detailed information in the following and in Example 1). In an embodiment, act b) includes determining the presence of a mutation in at least two or more genes selected from the group of Table 4, and wherein the presence of a mutation in at least two genes is indicative of a resistance to an antibiotic drug.

Instead of testing only single genes or mutants, a combination of several variant positions may improve the prediction accuracy and further reduce false positive findings that are influenced by other factors. Therefore, the presence of a mutation in 2, 3, 4, 5, 6, 7, 8 or 9 (or more) genes selected from Table 4 may be determined.

In a further embodiment, the present method includes in act b) determining the presence of a mutation in at least one gene selected from the group of genes listed in Table 5, and wherein the presence of a mutation in the at least one gene is indicative of a resistance to an antibiotic drug.

The genes according to Table 5 have never been described before in the context of antibiotic resistance of E. coli bacteria. They may be used for the determination of an antibiotic drug resistance of E. coli alone or in combination with other genes disclosed herein.

TABLE 5

| | | | | | |
|---|---|---|---|---|---|
| abgB | yegI | ymdC | ycjX | ldcA | yhjE |
| frwC | yehM | ynbB | ydiU | lplA | yjjG |
| hofB | yeiI | yncG | yfbL | menB | |
| htrL | yfaW | yneK | yfiK | metH | |
| hybA | yfcO | yphG | ygcR | pbpC | |
| hyfB | yfdF | zraS | ygcU | purH | |
| hyfI | yfdR | agaD | ygfZ | purL | |
| lsrF | yfdX | chbG | ygiF | queF | |
| potB | ygbN | eutE | ygjM | rhaB | |
| puuC | ygcQ | eutQ | yjjU | rplO | |
| yafT | ygeK | flgL | yjjW | srlD | |
| yagR | ygeO | gcvP | yohG | thiC | |
| yaiO | ygiD | gspO | yqjA | thiE | |
| ybbB | yhaC | gudD | yrfB | thiM | |
| ybfD | yhgE | hemF | ytfG | uxaA | |
| ybfQ | yhiJ | ldrA | aspS | ybiB | |
| ycbF | yjbI | livG | cysD | ybiU | |
| ycbS | yjcF | murP | eutA | ydfI | |
| ycgB | yjfF | nepI | fadA | ydgA | |
| ycgK | yjfZ | pphB | fdx | yecA | |
| yciQ | yjgL | ptrB | fhuC | yehT | |
| yddV | yjgN | rhaD | gudP | yfcN | |
| ydjO | yjhS | tiaE | helD | yheN | |
| yeaX | yjjJ | torZ | hrpB | yhgF | |
| yeeJ | ynfA | uidB | kdpD | yhhQ | |

For E. coli, 86 ultra highly significant pairs of genetic positions and drug resistance (Table 2) were identified. The 86 combinations correspond to 35 genetic positions, since the sites may be significant for more than one single drug. Most importantly, the respective sites are located in 9 genes: hofB, allA, mukB, ymdC, potB, ycgK, ycgB, valS, yjjJ. These genes thus appear to be critical for antibiotic resistance/susceptibility. The identified mutations all lead to amino acid alterations, either to an exchange of amino acid at the respective position or the creation of a new stop-codon. For more detailed information, it is referred to Example 1, below.

One embodiment relates to a method of determining the resistance or susceptibility of a bacterial microorganism belonging to the species E. coli to an antibiotic drug including: providing a sample containing or suspected of containing the bacterial microorganism belonging to the species E. coli; determining from the sample a nucleic acid sequence information of at least one gene selected from the group of hofB, allA, mukB, ymdC, potB, ycgK, ycgB, valS, and yjjJ; and based on the determination of the genetic information determining the resistance or susceptibility to the antibiotic drug.

In a further embodiment, the presence of a mutation in at least one gene selected from the group of hofB, allA, mukB, ymdC, potB, ycgK, ycgB, valS, and yjjJ is determined. Thus, the presence of a mutation in at least one or 2, 3, 4, 5, 6, 7, 8 or 9 of these genes may be analyzed.

In a further embodiment, the presence of a mutation in at least one gene selected from the group of the following table 6 is determined. The exact amino acid exchange indicated in Table 6 may be determined.

TABLE 6

| Gene | Amino Acid |
|---|---|
| Name | Exchange |
| aspS | D382E |
| birA | Q113H |
| cysD | D232N |
| dapB | N87K |
| dxs | A541T |
| eutA | A210V |
| fadA | V387I |
| fdx | S66T |
| fhuB | G448V |
| fhuC | A122V |
| fhuD | D76E |
| fmt | V30I |
| gudP | A448V |
| gyrA | D87N; D87Y |
| helD | E671D |
| hrpB | A413T |
| hrpB | V240A |
| ilvA | D401E |
| kdpD | E376D |
| ldcA | R167Q |
| lplA | A279T |
| menB | T31A |
| metH | E1124; E1124D |
| mukB | S1015N |
| parC | S80I |
| parC | S80R |
| pbpC | H37Q |
| purH | T366I |
| purK | N137D |
| purL | D615E |
| queF | K126E |
| rhaA | S406N |
| rhaB | T407A |
| rplO | K39N |
| srlD | M54T |
| thiC | H193R |
| thiE | A121E |
| thiE | R43Q |
| thiM | A122T |
| trpC | L378F |
| udp | I147M |
| uxaA | E236A |
| ybiB | G35S |
| ybiU | M419I |
| ydfI | A146V |
| ydgA | F416L |
| yecA | I195V |
| yehT | A106V |
| yfcN | I39V |
| yheN | Q49H |
| yhgF | E737D |
| yhhQ | R138H |
| yhjE | I323V |
| yjjG | A57V |
| ynfA | T84S |

Surprisingly, it was discovered that an overlap of mutations in functionally similar proteins of E. coli and K. pneumoniae exists. Interestingly, when considering the proteins that were associated significantly with at least one drug, an overlap of 1,746 proteins was found (same official name and more than 80 percent positives in BLAST in pairwise comparison) that are affected in E. coli as well as in K. pneumoniae. Extending the analysis to the exact AA exchanges in these proteins, an overlap of 55 mutated positions that are equal in both organisms were detected. Therefore, the above genes might form a valuable basis for the determination of the antibiotic resistance pattern in both, E. coli and K. pneumonia microorganisms.

According to an optional aspect, the nucleic acid sequence information may be the determination of the presence of a single nucleotide at a single position in at least one gene.

Thus the embodiments include a method wherein the presence of a single nucleotide polymorphism or mutation at a single nucleotide position is detected.

For example, this may be done in at least one gene selected from the group of hofB, allA, mukB, ymdC, potB, ycgK, ycgB, valS, and yjjJ. Therefore, according to an optional aspect, the mutation is a mutation selected from the group of mutations listed in table 2 (see below in Example 1). The present embodiments thus also include a method of determining an antibiotic resistance profile for a bacterial microorganism belonging to the species E. coli including the acts of a) providing a sample containing or suspected of containing the bacterial microorganism; b) determining the presence of a mutation in at least one position as identified in Table 2; wherein the presence of a mutation is indicative of a resistance to an antibiotic drug.

The determination may be made based on 1, 2, 3, 4, 5, 6, 7, and up to the 35 genetic positions identified in Table 2.

The method may include determining the resistance of E. coli to one or more antibiotic drugs. These drugs include, but are not restricted to antibiotic drugs selected from the group consisting of ampicillin sulbactam (A.S.), ampicillin (AM), amoxicillin clavulanate (AUG), aztreonam (AZT), ceftriaxone (CAX), ceftazidime (CAZ), cefotaxime (CFT), cefepime (CPM), ciprofloxacin (CP), ertapenem (ETP), levofloxacin (LVX), cefuroxime (CRM), piperazillin tazobactam (P/T), trimethoprim sulfamethoxazole (T/S), tobramycin (TO), gentamicin (GM), cefazolin (CFZ), cephalotin (CF), imipenem (IMP), meropenem (MER) and tetracycline (TE). See also Table 1.

It was discovered that mutations in certain genes are indicative not only for a resistance to one single antibiotic drug, but to groups containing several drugs.

For example, it turned out that in case of the group of lactam antibiotics, the presence of a mutation in the following genes: chbG, eutQ, flgL, gudD, gyrA, ldrA, menE, murB, murP, nepI, parC, pphB, ptrB, rhaD, ydiU, yegE, yegI, yfbL, yfiK, ygcR, ygiF, ygjM, yohG, and/or yrfB may be determined and is indicative for the presence of a resistance against antibiotics of this group.

The group of lactam antibiotics may include A.S., AM, AUG, AZT, CFZ, CPE, CFT, CAZ, CAX, CRM, CF, CP, IMP, MER, ETP and/or P/T. The p-value threshold for these identified genes is ≦10⁻⁴⁵.

It is within the scope of the present embodiments that the above determination is done based on a single gene or 2, 3, 4, etc. genes of this group, however, a mutation may be determined in all of these genes in relation to the reference strain K12 substrain DH10B (see also below for further information).

In a further embodiment, the antibiotic drug is selected from quinolone or aminoglycoside antibiotics and the presence of a mutation in the following genes is determined: agaD, chbG, eutE, eutQ, gcvP, gspO, gyrA, livG, menE, nepI, parC, speC, tiaF, torZ, uidB, yegE, yegI, yejA, ygcU, ygfZ, ygiF, ygjM, yjjU, yjjW, ymdC, ypdB, yqjA, and/or ytfG.

The quinolone and aminoglycoside antibiotics may be selected from CP, LVX, GM and TO.

Surprisingly, the relevant genes completely overlapped regarding a resistance to quinolone and aminoglycoside antibiotics; the p-value threshold for these genes is ≦10⁻⁵³. Also here, it is within the scope of the present embodiments that the determination is done based on a single gene or in 2, 3, 4, or more genes of this group only, however, a mutation may be determined in all of these genes in relation to the reference strain K12 substrain DH10B.

In a further embodiment, the antibiotic drug is selected from tetracycline and the presence of a mutation in at least one or more of the following genes is determined: astE, chbG, eutQ, flgL, gudD, gyrA, hemF, hypF, kdpE, ldrA, menE, murB, murP, nepI, ompC, parC, pphB, ptrB, and/or rhaD. The p-value threshold is ≦10⁻⁴⁷.

In a still further embodiment, the antibiotic drug is selected from trimethoprim sulfmethoxazol and the presence of a mutation in at least one or more of the following genes is determined: astE, chbG, eutQ, flgL, gudD, gyrA, ldrA, menE, murB, nepI, parC, ycjX, ydiU, yegE, yfiK, ygcR, ygiF, and/or yrfB. The p-value threshold is ≦10⁻⁴⁸.

In an embodiment, the method includes determining a mutation, wherein the mutation is selected from the group of mutations listed in Table 7. Table 7 is depicted in the following:

TABLE 7

| Genome Pos | Therapy | Ref | Alt | AA | Alt AA | Gene | Exchange |
|---|---|---|---|---|---|---|---|
| 37032 | P/T | C | T | V | I | caiC | V270I |
| 206427 | AM | C | A, G | T | R, K | yafE | T133R; T133K |
| 319290 | P/T | A | T | D | E | yaiO | D36E |
| 1181357 | AM | C | T | T | M | yceH | T178M |
| 1368519 | P/T | G | T | AA | S | — | A137S |
| 1516808 | P/T | G | A, T | AA | T, S | stfR | A114T; A114S |
| 1517573 | P/T | G | C | E | Q | stfR | E369Q |
| 1567286 | P/T | G | A | AA | T | ynbB | A148T |
| 1615473 | AZT, CAX | A | C, T | E | V, A | yncG | E203V; E203A |
| 1684413 | AM | C | T | M | I | ydeK | M441I |
| 1974644 | A/S | T | A, C | C | R, S | yeaX | C69R; C69S |
| 2052365 | ETP | A | T, C | I | Stop | flhA | I427; I427M |
| | | | | | codon, M | | |
| 2178525 | P/T | C | T | G | D | yefM | G74D |
| 2216164 | P/T | C | T, G | R | K, T | fcl | R20K; R20T |
| 2233638 | ETP, P/T | G | A, T | L | F, Stop | yegE | L447F; L447 |
| | | | | | codon | | |
| 2428172 | CP, LVX | C | A, T | D | N, Y | gyrA | D87N; D87Y |
| 2428183 | A/S, AM, | G | A | S | L | gyrA | S83L |
| | AZT, CAX, | | | | | | |
| | CAZ, CFT, | | | | | | |
| | CPE, CRM, | | | | | | |
| | GM, T/S, | | | | | | |
| | TO | | | | | | |
| 2463877 | AUG | A | G | V | A | menE | V46A |
| 2565236 | ETP | G | T, A | A | S, T | yfdR | A156S; A156T |
| 2725302 | P/T | G | A | M | I | xseA | M428I |
| 2755319 | P/T | T | C | M | T | csiE | M33T |
| 2924554 | P/T | A | T | T | S | norW | T27S |
| 3240296 | P/T | G | A, C | F | Stop | hybA | F204; F204L |
| | | | | | codon, L | | |
| 4054212 | A/S | C | A, T | E | Stop | ilvY | E184; E184D |
| | | | | | codon, D | | |
| 4525576 | AM | T | C | I | V | yjfZ | I78V |
| 4553471 | ETP | C | A, T | L | I, F | yjfF | L20I; L20F |
| 4575887 | P/T | T | C, G | L | R, P | yjgL | L207R; L207P |
| 4636902 | AM | G | A | A | V | — | A175V |

A part from the above genes indicative of a resistance against antibiotics, single nucleotide polymorphisms (=SNP's) may have a high significance for the presence of a resistance against defined antibiotic drugs. The analysis of these polymorphisms on a nucleotide level may further improve and accelerate the determination of a drug resistance to antibiotics in E. coli.

For example, a resistance of E. coli against the antibiotic drug AM may be determined by the presence of a single nucleotide polymorphism in at least one, for example, 1, 2, 3, 4, 5, or 6 of the following nucleotide positions: 2428183, 4525576, 1684413, 4636902, 1181357, 206427.

In an embodiment, the antibiotic drug is A/S and an SNP in at least one, for example, 1, 2 or 3 of the following nucleotide positions is detected: 2428183, 4054212, 1974644.

In a further embodiment, the antibiotic drug is AUG and a mutation in the following nucleotide position is detected: 2463877.

For a resistance to the antibiotic drug AZT, a mutation in at least one of the following nucleotide positions is detected: 2428183, 1615473.

In a still further embodiment, the antibiotic drug is CAX and a mutation in at least one of the following nucleotide positions is detected: 2428183, 1615473.

A resistance to the antibiotic drugs CFT, CP, CPE, CRM, GM, LVX, TO, T/S or CAZ may be detected by a mutation in the nucleotide position 2428183.

When the antibiotic drug is ETP, a mutation in at least one, for example 1, 2, 3, or 4 of the following nucleotide positions is detected: 2052365, 2233638, 4553471, 2565236.

In a further embodiment, the antibiotic drug is P/T and a mutation in at least one, for example, 1, 2, 3, 4, 5, 6, 7, 8, 9, 10, 11, 12, 13, or 14 of the following nucleotide positions is detected: 2233638, 2216164, 2725302, 1567286, 2755319, 319290, 3240296, 1517573, 2178525, 2924554, 1516808, 37032, 1368519, 4575887.

The resistance to the respective antibiotic drug may be tested according to the decision diagrams of FIGS. 5-20.

A decision diagram or “decision tree” is a tree-like graph for prediction tasks, e.g., classification. Given a data set including a number of samples with feature values (e.g., any measurements, here SNPs) and class labels (e.g., resistant/not resistant against a certain drug), a decision tree models the decision process of inferring the sample class label from its feature values.

To build the model a given data set is used (as described above): Among all features (SNPs) and their possible values (DNA bases A, C, T, and G) the feature value is selected that achieves the optimal sample separation with respect to the given sample labels. That may be a SNP whose value for not resistant samples would be different as for the resistant samples. The selected feature value becomes the root of the tree (e.g., the first tree node, often drawn at the top) and the samples are split according to that feature, e.g., samples having that feature value and samples with another value. The resulting subsets of samples form new nodes and the feature selection and splitting process is repeated for each of them separately. This procedure stops if a specific criterion is fulfilled (e.g., no further improvement or maximal tree size is achieved).

The used graphical representation is defined as follows:

The tree root is drawn at the top. Each node contains following information: (1) Its feature and its value(s) drawn below the node, e.g. SNP 2428183=G. (2) Class label: 0=not resistant, 1=resistant. (3) Class distribution: The proportion of samples contained in that node belonging to class 0 or 1. (4) Proportion of samples contained in that node (w.r.t. to sample number used to build the tree). (5) Color: green=0, blue=1, the stronger the color the higher the certainty for the chosen class label.

The model may be built on the so-called training set and its prediction power may be tested on the so-called test set (e.g., to assess the model performance on unseen data). Both data sets may be independent and have no intersection. However, if the available data set is not large enough to form a sufficient large training and test data sets, we apply a procedure called k-fold cross validation (CV): We divide our data set into k subsets of equal size, then each of the k subsets is used once as test data and the rest as training data. The final tree is built on the whole data set, so the CV is only used to estimate the performance of the final model.

The classification of a new sample works as follows: (1) One starts at the tree root: the value of the root attribute in the sample is checked. If the value is equal to the root value then one goes left to the next node. Otherwise, one goes right. (2) The value of the current node attribute in the sample is checked and it is decided again whether to go left or right. And so on. (3) The process stops if one is in a leaf node (terminal node, node without outgoing edges). The sample gets the same label as that leaf node.

According to an optional aspect, a detected mutation is a mutation leading to an altered amino acid sequence in a polypeptide derived from a respective gene in which the detected mutation is located. According to this aspect, the detected mutation thus leads to a truncated or version of the polypeptide (wherein a new stop codon is created by the mutation) or a mutated version of the polypeptide having an amino acid exchange at the respective position.

According to an optional aspect, determining the nucleic acid sequence information or the presence of a mutation includes determining a partial sequence or an entire sequence of the at least one gene.

According to an optional aspect, determining the nucleic acid sequence information or the presence of a mutation includes determining a partial or entire sequence of the genome of the bacterial microorganism, wherein the partial or entire sequence of the genome includes at least a partial sequence of the at least one gene.

According to an optional aspect, the sample is a patient sample (clinical isolate).

According to an optional aspect, determining the nucleic acid sequence information or the presence of a mutation includes a using a next generation sequencing or high throughput sequencing method. According to a further aspect, a partial or entire genome sequence of the bacterial organism is determined by a using a next generation sequencing or high throughput sequencing method.

According to an optional aspect, the method further includes determining the resistance to 2, 3, 4, 5, or 6 antibiotic drugs.

In a further aspect, the present embodiments are directed to a diagnostic method of determining an antibiotic resistant E. coli infection in a patient, including the acts of: a) obtaining or providing a sample containing or suspected of containing E. coli from the patient; and b) determining the presence of at least one mutation in at least one gene as described above, wherein the presence of the at least one mutation is indicative of an antibiotic resistant E. coli infection in the patient.

In a still further aspect, the present embodiments are directed to a method of treating a patient suffering from an antibiotic resistant E. coli infection in a patient: a) obtaining or providing a sample containing or suspected of containing E. coli from the patient; b) determining the presence of at least one mutation in at least one gene as described above, wherein the presence of the at least one mutation is indicative of a resistance to one or more antibiotic drugs; c) identifying the at least one or more antibiotic drugs; d) selecting one or more antibiotic drugs different from the ones identified in act c) and being suitable for the treatment of an E. coli infection; and e) treating the patient with the one or more antibiotic drugs.

According to an embodiment, the patient is a vertebrate, e.g., a mammal such as a human patient.

Regarding the dosage of the antibiotic drug, it is referred to the established principles of pharmacology in human and veterinary medicine. For example, Forth, Henschler, Rummel “Allgemeine und spezielle Pharmakologie und Toxikologie”, 9th edition, 2005 might be used as a guideline. Regarding the formulation of a ready-to-use medicament, reference is made to “Remington, The Science and Practice of Pharmacy”, 22^{nd} edition, 2013.

### DEFINITIONS

Unless defined otherwise, technical and scientific terms used herein have the same meaning as commonly understood by one of ordinary skill in the art.

The term “nucleic acid molecule” refers to a polynucleotide molecule having a defined sequence. It includes DNA molecules, RNA molecules, nucleotide analog molecules and combinations and derivatives thereof, such as DNA molecules or RNA molecules with incorporated nucleotide analogs or cDNA.

The term “nucleic acid sequence information” relates to an information that may be derived from the sequence of a nucleic acid molecule, such as the sequence itself or a variation in the sequence as compared to a reference sequence.

The term “mutation” relates to a variation in the sequence as compared to a reference sequence. Such a reference sequence may be a sequence determined in a predominant wild type organism or a reference organism, e.g., a defined and known bacterial strain or substrain. A mutation is, for example, a deletion of one or multiple nucleotides, an insertion of one or multiple nucleotides, or substitution of one or multiple nucleotides, duplication of one or a sequence of multiple nucleotides, translocation one or a sequence of multiple nucleotides, and, in particular, a single nucleotide polymorphism (SNP).

As used herein, a “sample” is a sample including nucleic acid molecule from a bacterial microorganism. Examples for samples are: cells, tissue, body fluids, biopsy specimens, blood, urine, saliva, sputum, plasma, serum, cell culture supernatant, swab sample, and others.

New and highly efficient methods of sequencing nucleic acids referred to as next generation sequencing have opened the possibility of large scale genomic analysis. The term “next generation sequencing” or “high throughput sequencing” refers to high-throughput sequencing technologies that parallelize the sequencing process, producing thousands or millions of sequences at once. Examples include Massively Parallel Signature Sequencing (MPSS) Polony sequencing, 454 pyrosequencing, Illumina (Solexa) sequencing, SOLiD sequencing, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing.

It is to be understood that this invention is not limited to the particular component parts of the process acts of the methods described herein as such methods may vary. It is also to be understood that the terminology used herein is for purposes of describing particular embodiments only, and is not intended to be limiting. It is noted that, as used in the specification and the appended claims, the singular forms “a,” “an” and “the” include singular and/or plural referents unless the context clearly dictates otherwise. For example, the term “a” as used herein may be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1 depicts an exemplary contingency table for the computation of the Fisher's exact test and the measures accuracy, sensitivity, specificity, positive predictive value (PPV), and negative predictive value (NPV). Numbers are given for amino acid exchange S83L (GyrA) and Ciprofloxacin.

FIG. 2 depicts an overview of mean MIC values for Ciprofloxacin for samples having no mutation in GyrA (S83, D87) and ParC (S80), either one mutation in GyrA and not ParC, both mutations in GyrA and not ParC, or all three mutations.

FIG. 3 depicts Panel A: bar chart of genes with highest number of significant sites, Panel B: bar chart detailing the genes with highest number of sites correlated to at least 3 drugs, and Panel C: Scatter plot showing for each gene the number of significant sites correlated with at least 3 drugs as function of total number of significant sites in the gene. Colored genes represent those with highest absolute numbers (yfaL, yehL, YjgL)), with higher frequency of resistance correlated to at least 3 drugs (yjgN) and genes with lower significant sites in at least 3 drugs (fhuA, yeeJ). Panel D. Along gene plot for yigN. The significant sites along the genetic sequence are presented as dots, the y-axis shows the number of drug classes significant for the respective site. Below, a so called snake plot of the trans-membrane protein is shown, the affected amino acids are indicated.

FIG. 4 depicts Panel A: network diagram showing drugs as rectangles and genes with higher or lower coverage if resistance for the respective drug is shown as circles. mmuP, mmuM, yiel, insN-1 correspond to higher read counts in case of resistant isolates while green genes correspond to lower coverage, and Panel B and C: two example along-chromosome plots. Each sample is represented by a line, black lines correspond to non-resistant and gray lines to resistant isolates.

FIG. 5 depicts a decision diagram for ampicillin.

FIG. 6 depicts a decision diagram for ampicillin sulbactam.

FIG. 7 depicts a decision diagram for amoxicillin clavulanate.

FIG. 8 depicts a decision diagram for aztreonam.

FIG. 9 depicts a decision diagram for ceftriaxone.

FIG. 10 depicts a decision diagram for ceftazidime.

FIG. 11 depicts a decision diagram for cefotaxime.

FIG. 12 depicts a decision diagram for ciprofloxacin.

FIG. 13 depicts a decision diagram for cefepime.

FIG. 14 depicts a decision diagram for cefuroxime.

FIG. 15 depicts a decision diagram for ertapenem.

FIG. 16 depicts a decision diagram for gentamycin.

FIG. 17 depicts a decision diagram for levofloxacin.

FIG. 18 depicts a decision diagram for piperazillin tazobactam.

FIG. 19 depicts a decision diagram for tobramycin.

FIG. 20 depicts a decision diagram for trimethoprim sulfmethoxazole.

### EXAMPLES

### Example 1

Here, a unique collection of genes was identified that allow the determination the resistance of a bacterial microorganism to commonly used antibiotic drugs.

A unique cohort of bacterial samples obtained from 150 clinical isolates was sequenced in order to understand the genetic resistance mechanisms by using High Throughput sequencing. In parallel, classical resistance tests were applied using 21 drugs or combinations of drugs (Table 1).

TABLE 1
Antibiotic Drugs

| Medication | Drugbank ID | Abbreviation |
|---|---|---|
| Amoxicillin | DB00766 | AUG |
| Clavulanate | DB01060 | |
| Ampicillin | DB00415 | AM |
| Ampicillin Sulbactam | DB00415 | A/S |
| Aztreonam | DB00355 | AZT |
| Cefazolin | DB01327 | CFZ |
| Cefepime | DB01413 | CPE |
| Cefotaxime | DB00493 | CFT |
| Ceftazidime | DB00438 | CAZ |
| Ceftriaxone | DB01212 | CAX |
| Cefuroxime | DB01112 | CRM |
| Cephalotin | DB00456 | CF |
| Ciprofloxacin | DB00537 | CP |
| Gentamicin | DB00798 | GM |
| Imipenem | DB01598 | IMP |
| Levofloxacin | DB01137 | LVX |
| Piperacillin | DB00319 | P/T |
| Tazobactam | DB01606 | |
| Tetracycline | DB00759 | TE |
| Tobramycin | DB00684 | TO |
| Trimethoprim | DB00440 | T/S |
| Sulfamethoxaxole | DB01015 | |
| Meropenem | DB00760 | MER |
| Ertapenem | DB00303 | ETP |

E. coli strains to be tested were seeded on agar plates and incubated under growth conditions for 24 hours. Then, colonies were picked and incubated in growth medium in the presence of a given antibiotic drug in dilution series under growth conditions for 16-20 hours. Bacterial growth was determined by observing turbidity.

Next mutations were searched that are highly correlated with the results of the phenotypic resistance test.

For sequencing, samples were prepared using a Nextera library preparation, followed by multiplexed sequencing using the Illuminat HiSeq 2500 system, paired end sequencing. Data were mapped with BWA (Li H. and Durbin R. (2010) Fast and accurate long-read alignment with Burrows-Wheeler Transform. Bioinformatics, Epub. [PMID: 20080505]) and SNP were called using samtools (Li H.*, Handsaker B.*, Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9. [PMID: 19505943]).

The reference sequence was obtained from Escherichia coli str. K-12 substr.

### DH10B:

LOCUS CP000948 4686137 bp DNA circular BCT 5 Jun. 2008
DEFINITION Escherichia coli str. K12 substr. DH10B, complete genome.

### ACCESSION CP000948

### VERSION CP000948.1 GI:169887498

### DBLINK BioProject: PRJNA20079

### KEYWORDS .

SOURCE Escherichia coli str. K-12 substr. DH10B

ORGANISM Escherichia coli str. K-12 substr. DH10B

Bacteria; Proteobacteria; Gammaproteobacteria; Enterobacteriales; Enterobacteriaceae; Escherichia.

REFERENCE 1 (bases 1 to 4686137)
AUTHORS Durfee, T., Nelson, R., Baldwin, S., Plunkett, G. III, Burland, V., Mau, B., Petrosino, J. F., Qin, X., Muzny, D. M., Ayele, M., Gibbs, R. A., Csorgo, B., Posfai, G., The inventors in stock, G. M. and Blattner, F. R.
TITLE The complete genome sequence of Escherichia coli DH10B: insights into the biology of a laboratory workhorse
JOURNAL J. Bacteriol. 190 (7), 2597-2606 (2008)
PUBMED 18245285
   REFERENCE 2 (bases 1 to 4686137)
AUTHORS Plunkett, G. III.
TITLE Direct Submission
JOURNAL Submitted (20 Feb. 2008) Department of Genetics and Biotechnology, University of Wisconsin, 425G Henry Mall, Madison, Wis. 53706, USA
COMMENT DH10B and DH10B-T1R are available from Invitrogen Corporation (http://www.invitrogen.com).

The mutations were matched to the genes and the amino acid changes were calculated. Using different algorithms (SVM, homology modeling) mutations leading to amino acid changes with likely pathogenicity/resistance were calculated. Known variants from the swissprot database were excluded and all variants in the respective genes selected.

As noted above, for E. coli 86 ultra highly significant pairs of genetic positions and drug resistance (Table 2) were identified. The 86 combinations correspond to 35 genetic positions, since the sites may be significant for more than one single drug. Most importantly, the respective sites are located in 9 genes: hofB, allA, mukB, ymdC, potB, ycgK, ycgB, valS, yjjJ. These genes thus appear to be critical for antibiotic resistance/susceptibility. The identified mutations all lead to amino acid alterations, either to an exchange of amino acid at the respective position or the creation of a new stop-codon. Thereby, resistance related variants for the following 6 antibiotic drugs were detected: CP, LVX, TE, CFZ, CRM, GM.

TABLE 2
Identified Mutations

| Genome | | | | | | | Alt | | |
|---|---|---|---|---|---|---|---|---|---|
| Pos | Therapy | p-value | gene pos | Ref | Alt | AA | AA | Gene | Exchange |
| 90064 | CP | 2.6527E−22 | 1140 | C | T | W | C | hofB | W380C |
| 90064 | LVX | 5.2992E−20 | 1140 | C | T | W | C | hofB | W380C |
| 471036 | CP | 2.6527E−22 | 31 | G | A | E | K | allA | E11K |
| 471036 | LVX | 5.2992E−20 | 31 | G | A | E | K | allA | E11K |
| 1030161 | CP | 2.6527E−22 | 685 | C | T | R | C | mukB | R229C |
| 1030161 | LVX | 5.2992E−20 | 685 | C | T | R | C | mukB | R229C |
| 1161719 | CP | 4.7722E−13 | 697 | A | C | N | H | ymdC | N233H |
| 1161719 | LVX | 8.8353E−13 | 697 | A | C | N | H | ymdC | N233H |
| 1161764 | CP | 2.6527E−22 | 742 | C | T | R | C | ymdC | R248C |
| 1161764 | LVX | 5.2992E−20 | 742 | C | T | R | C | ymdC | R248C |
| 1238314 | CP | 3.4979E−18 | 799 | A | G | L | V | potB | L267V |
| 1238314 | LVX | 7.91E−17 | 799 | A | G | L | V | potB | L267V |
| 1238314 | TE | 2.5459E−07 | 799 | A | G | L | V | potB | L267V |
| 1239076 | CP | 2.6527E−22 | 37 | C | T | V | F | potB | V13F |
| 1239076 | LVX | 5.2992E−20 | 37 | C | T | V | F | potB | V13F |
| 1266748 | CP | 8.9009E−16 | 189 | A | G | H | Q | ycgK | H63Q |
| 1266748 | LVX | 1.2635E−14 | 189 | A | G | H | Q | ycgK | H63Q |
| 1266748 | CFZ | 4.4331E−10 | 189 | A | G | H | Q | ycgK | H63Q |
| 1266748 | TE | 6.1345E−10 | 189 | A | G | H | Q | ycgK | H63Q |
| 1266748 | CRM | 6.0841E−07 | 189 | A | G | H | Q | ycgK | H63Q |
| 1266829 | CP | 2.6527E−22 | 108 | G | A | S | R | ycgK | S36R |
| 1266829 | LVX | 5.2992E−20 | 108 | G | A | S | R | ycgK | S36R |
| 1275217 | LVX | 1.2048E−15 | 1489 | T | C | I | L | ycgB | I497L |
| 1275217 | CP | 1.5912E−15 | 1489 | T | C | I | L | ycgB | I497L |
| 1275217 | TE | 1.8029E−07 | 1489 | T | C | I | L | ycgB | I497L |
| 1275217 | CFZ | 6.7512E−07 | 1489 | T | C | I | L | ycgB | I497L |
| 1275307 | CP | 2.6527E−22 | 1399 | G | A | L | M | ycgB | L467M |
| 1275307 | LVX | 5.2992E−20 | 1399 | G | A | L | M | ycgB | L467M |
| 4582262 | CP | 3.7422E−11 | 1407 | G | A | D | E | valS | D469E |
| 4582262 | LVX | 7.4232E−10 | 1407 | G | A | D | E | valS | D469E |
| 4582262 | CFZ | 7.6762E−07 | 1407 | G | A | D | E | valS | D469E |
| 4582280 | CP | 1.1193E−12 | 1389 | A | G | D | E | valS | D463E |
| 4582280 | LVX | 1.2386E−12 | 1389 | A | G | D | E | valS | D463E |
| 4582301 | LVX | 1.6858E−16 | 1368 | T | C | K | N | valS | K456N |
| 4582301 | CP | 4.0168E−16 | 1368 | T | C | K | N | valS | K456N |
| 4582313 | LVX | 3.6594E−16 | 1356 | G | A | D | E | valS | D452E |
| 4582313 | CP | 2.3782E−15 | 1356 | G | A | D | E | valS | D452E |
| 4582337 | CP | 3.3749E−11 | 1332 | G | A | N | K | valS | N444K |
| 4582337 | LVX | 1.2081E−10 | 1332 | G | A | N | K | valS | N444K |
| 4582337 | CFZ | 3.6214E−08 | 1332 | G | A | N | K | valS | N444K |
| 4582352 | CP | 4.0453E−16 | 1317 | A | G | Y | * | valS | Y439* |
| 4582352 | LVX | 5.235E−16 | 1317 | A | G | Y | * | valS | Y439* |
| 4582352 | CFZ | 3.2243E−07 | 1317 | A | G | Y | * | valS | Y439* |
| 4582382 | CP | 3.7422E−11 | 1287 | C | T | L | F | valS | L429F |
| 4582382 | LVX | 7.4232E−10 | 1287 | C | T | L | F | valS | L429F |
| 4582430 | CP | 1.4858E−11 | 1239 | G | A | Y | * | valS | Y413* |
| 4582430 | LVX | 4.4757E−11 | 1239 | G | A | Y | * | valS | Y413* |
| 4582430 | CFZ | 2.3492E−07 | 1239 | G | A | Y | * | valS | Y413* |
| 4582838 | LVX | 1.1423E−14 | 831 | T | C | K | N | valS | K277N |
| 4582838 | CP | 1.2258E−14 | 831 | T | C | K | N | valS | K277N |
| 4582838 | CFZ | 7.2547E−08 | 831 | T | C | K | N | valS | K277N |
| 4582937 | LVX | 6.011E−18 | 732 | A | G | D | E | valS | D244E |
| 4582937 | CP | 2.912E−17 | 732 | A | G | D | E | valS | D244E |
| 4582937 | GM | 7.6578E−07 | 732 | A | G | D | E | valS | D244E |
| 4582943 | CP | 1.7589E−13 | 726 | G | A | Y | * | valS | Y242* |
| 4582943 | LVX | 1.3467E−11 | 726 | G | A | Y | * | valS | Y242* |
| 4582943 | CFZ | 1.2817E−09 | 726 | G | A | Y | * | valS | Y242* |
| 4582943 | TE | 1.3866E−07 | 726 | G | A | Y | * | valS | Y242* |
| 4582987 | CP | 1.7363E−22 | 682 | G | A | L | M | valS | L228M |
| 4582987 | LVX | 3.2E−21 | 682 | G | A | L | M | valS | L228M |
| 4583141 | CP | 3.6032E−18 | 528 | A | G | D | E | valS | D176E |
| 4583141 | LVX | 1.1539E−17 | 528 | A | G | D | E | valS | D176E |
| 4583141 | CFZ | 7.2389E−08 | 528 | A | G | D | E | valS | D176E |
| 4666362 | CP | 5.4173E−19 | 109 | G | A | D | N | yjjJ | D37N |
| 4666362 | LVX | 2.3023E−18 | 109 | G | A | D | N | yjjJ | D37N |
| 4666405 | CP | 5.4173E−19 | 152 | C | T | A | V | yjjJ | A51V |
| 4666405 | LVX | 2.3023E−18 | 152 | C | T | A | V | yjjJ | A51V |
| 4666461 | CP | 9.4295E−08 | 208 | A | G | T | A | yjjJ | T70A |
| 4666461 | LVX | 8.4496E−07 | 208 | A | G | T | A | yjjJ | T70A |
| 4666768 | CP | 9.4295E−08 | 515 | A | G | H | R | yjjJ | H172R |
| 4666768 | LVX | 8.4496E−07 | 515 | A | G | H | R | yjjJ | H172R |
| 4666804 | CP | 1.6956E−22 | 551 | A | G | H | R | yjjJ | H184R |
| 4666804 | LVX | 7.7611E−22 | 551 | A | G | H | R | yjjJ | H184R |
| 4666804 | CFZ | 4.5083E−07 | 551 | A | G | H | R | yjjJ | H184R |
| 4666885 | CP | 9.4295E−08 | 632 | A | G | Y | C | yjjJ | Y211C |
| 4666885 | LVX | 8.4496E−07 | 632 | A | G | Y | C | yjjJ | Y211C |
| 4667178 | CP | 9.4295E−08 | 925 | C | G | Q | E | yjjJ | Q309E |
| 4667178 | LVX | 8.4496E−07 | 925 | C | G | Q | E | yjjJ | Q309E |
| 4667191 | CP | 2.6527E−22 | 938 | G | A | R | H | yjjJ | R313H |
| 4667191 | LVX | 5.2992E−20 | 938 | G | A | R | H | yjjJ | R313H |
| 4667359 | CP | 9.4295E−08 | 1106 | T | C | V | A | yjjJ | V369A |
| 4667359 | LVX | 8.4496E−07 | 1106 | T | C | V | A | yjjJ | V369A |
| 4667424 | CP | 2.6527E−22 | 1171 | G | A | V | I | yjjJ | V391I |
| 4667424 | LVX | 5.2992E−20 | 1171 | G | A | V | I | yjjJ | V391I |
| 4667568 | CP | 1.2838E−17 | 1315 | G | A | A | T | yjjJ | A439T |
| 4667568 | LVX | 2.3051E−17 | 1315 | G | A | A | T | yjjJ | A439T |

In Table 2, the columns are designated as follows:

Genome Pos: genomic position of the SNP/variant in the E. coli reference genome (see below);
Therapy: the therapy to which the mutation is significantly correlated, multiple therapies are in separate rows (if a SNP is correlated to e.g. 4 therapies this leads to 4 single rows);
P-value: significance value calculated using fishers exact test;
Gene pos: position of the mutation in the gene;
Ref: reference base, A, C, T, G;
Alt: Alternative base associated with resistance;
AA: original Amino acid;
Alt A: changed amino acid;
Gene: affected gene;
Exchange: amino acid exchange in standard nomenclature;
P-value was calculated using the fisher exact test based on contingency table with 4 fields: #samples Resistant/wild type; #samples Resistant/mutant; #samples not Resistant/wild type; #samples not Resistant/mutant

In Table 3, the identified genes and gene products are listed and identified by Gene ID of the gene and (NCBI) Accession number of the corresponding protein corresponding to:

TABLE 3
Gene name and Identifier

| Gene name | Gene ID | Accession No. |
|---|---|---|
| hofB | 6061494 | ACB01286.1 |
| allA | 6059827 | ACB01630.1 |
| mukB | 6060547 | ACB02124.1 |
| ymdC | 6059214 | ACB02240.1 |
| potB | 6058608 | ACB02318.1 |
| ycgK | 6058586 | ACB02348.1 |
| valS | 6060190 | ACB05239.1 |
| yjjJ | 6058313 | ACB05313.1 |
| ycgB | 6058539 | ACB02358.1 |

The test is based on the distribution of the samples in the 4 fields. Even distribution indicates no significance, while clustering into two fields indicates significance.

Using this approach 35 highly significant, novel genetic positions or mutations in 9 genes (hofB, allA, mukB, ymdC, potB, ycgK, ycgB, valS, yjjJ) were identified that may be used for and allow the determination of resistance to commonly used antibiotic drugs. All the highly significant mutations described herein and listed in table 2 are non-conservative mutations leading to an amino acid exchange or a new stop-codon (designated with a “*” symbol in table 2), and thus to an altered protein. It is thus likely that the identified 9 genes play a significant role in antibiotic resistance and are putative targets for developing new drug candidates.

### Example 2

In this example, genetic susceptibility of E. coli to 21 different drugs from five drug classes is evaluated (see below).

Methods: Antimicrobial susceptibility test (AST) for 1,162 clinical E. coli isolates with varying spectra of resistance to 21 FDA-approved drugs was performed and genomes of all isolates were sequenced. Genetic variants were correlated to the AST data.

Results: 25,744 sites in the E. coli genome significantly correlated to drug resistance are reported. Highest significance was reached for the drugs Ciprofloxacin and Levofloxacin with respect to amino acid (AA) exchange S83L in GyrA (p_{Ciprofloxacin}=10⁻²³⁵ accuracy, specificity and sensitivity: 98%, 99%, and 94%; p_{Levofloxacin}=10⁻²⁰⁹, 97%, 98%, 93%), a target for quinolones. The second most significant association was observed for ParC, a second target of quinolones (AA exchange S80I, p_{Ciprofloxacin}=10⁻¹⁹⁶ and p_{Levofloxacin}=10⁻¹⁹⁴). Particularly many AA exchanges significantly associated with resistance to multiple drugs were discovered in YigN. By analyzing the sequence coverage on the genome level, a gene dose dependency of several genes is identified, including mmuP and mmuM, encoding a putative S-methylmethionine transporter and a homocysteine S-methyltransferase. Both loci are associated with resistance against â-lactams and quinolones.

Conclusion: a high-throughput screening and analysis pipeline is presented to investigate antibiotics resistance in E. coli strains. The results demonstrate the potential of genetics-based tests to predict susceptibility against antimicrobial drugs. In addition, novel correlations of gene dose to resistance are reported.

A systematic evaluation using E. coli was carried out. Specifically, 1,162 E. coli samples were collected over 22 years (1991-2013) across over 60 different institutes. For these isolates, the standard AST for 21 FDA-approved drugs was carried out and performed Whole Genome Sequencing (WGS) for the same 1,162 isolates to build a database revealing genetic sites for predicting AST from genetic data.

### Methods

### Bacterial Strains

1,162 E. coli strains are selected from the microbiology strain collection at Siemens Healthcare Diagnostics (West Sacramento, Calif.) for susceptibility testing and whole genome sequencing.

### Antimicrobial Susceptibility Testing Panels

Frozen reference AST panels were prepared following Clinical Laboratory Standards Institute (CLSI) recommendations′. The following antimicrobial agents (with μg/ml concentrations shown in parentheses) were included in the panels: Amoxicillin/K Clavulanate (0.5/0.25-64/32), Ampicillin (0.25-128), Ampicillin/Sulbactam (0.5/0.25-64/32), Aztreonam (0.25-64), Cefazolin (0.5-32), Cefepime (0.25-64), Cefotaxime (0.25-128), Ceftazidime (0.25-64), Ceftriaxone (0.25-128), Cefuroxime (1-64), Cephalothin (1-64), Ciprofloxacin (0.015-8), Ertepenem (0.12-32), Gentamicin (0.12-32), Imipenem (0.25-32), Levofloxacin (0.25-16), Meropenem (0.12-32), Piperacillin/Tazobactam (0.25/4-256/4), Tetracycline (0.5-64), Tobramycin (0.12-32), and Trimethoprim/Sulfamethoxazole (0.25/4.7-32/608). Prior to use with clinical isolates, AST panels were tested with QC strains. AST panels were considered acceptable for testing with clinical isolates when the QC results met QC ranges described by CLSI16.

### Inoculum Preparation

Isolates were cultured on trypticase soy agar with 5% sheep blood (BBL, Cockeysville, Md.) and incubated in ambient air at 35±1° C. for 18-24 h. Isolated colonies (4-5 large colonies or 5-10 small colonies) were transferred to a 3 ml Sterile Inoculum Water (Siemens) and emulsified to a final turbidity of a 0.5 McFarland standard. 2 ml of this suspension was added to 25 ml Inoculum Water with Pluronic-F (Siemens). Using the Inoculator (Siemens) specific for frozen AST panels, 5 μl of the cell suspension was transferred to each well of the AST panel. The inoculated AST panels were incubated in ambient air at 35±1° C. for 16-20 h. Panel results were read visually, and minimal inhibitory concentrations (MIC) were determined.

### DNA Extraction

Four streaks of each Gram-negative bacterial isolate cultured on trypticase soy agar containing 5% sheep blood and cell suspensions were made in sterile 1.5 ml collection tubes containing 50 μl Nuclease-Free Water (AM9930, Life Technologies). Bacterial isolate samples were stored at −20° C. until nucleic acid extraction. The Tissue Preparation System (TPS) (096D0382-02_01_B, Siemens) and the VERSANT® Tissue Preparation Reagents (TPR) kit (10632404B, Siemens) were used to extract DNA from these bacterial isolates. Prior to extraction, the bacterial isolates were thawed at room temperature and were pelleted at 2000 G for 5 seconds. The DNA extraction protocol DNAext was used for complete total nucleic acid extraction of 48 isolate samples and eluates, 50 μl each, in 4 hours. The total nucleic acid eluates were then transferred into 96-Well qPCR Detection Plates (401341, Agilent Technologies) for RNase A digestion, DNA quantitation, and plate DNA concentration standardization processes. RNase A (AM2271, Life Technologies), which was diluted in nuclease-free water following manufacturer's instructions, was added to 50 μl of the total nucleic acid eluate for a final working concentration of 20 μg/ml. Digestion enzyme and eluate mixture were incubated at 37° C. for 30 minutes using Siemens VERSANT® Amplification and Detection instrument. DNA from the RNase digested eluate was quantitated using the Quant-iT™ PicoGreen dsDNA Assay (P11496, Life Technologies) following the assay kit instruction, and fluorescence was determined on the Siemens VERSANT® Amplification and Detection instrument. Data analysis was performed using Microsoft® Excel 2007. 25 μl of the quantitated DNA eluates were transferred into a new 96-Well PCR plate for plate DNA concentration standardization prior to library preparation. Elution buffer from the TPR kit was used to adjust DNA concentration. The standardized DNA eluate plate was then stored at −80° C. until library preparation.

### Next Generation Sequencing

Prior to library preparation, quality control of isolated bacterial DNA was conducted using a Qubit 2.0 Fluorometer (Qubit dsDNA BR Assay Kit, Life Technologies) and an Agilent 2200 TapeStation (Genomic DNA ScreenTape, Agilent Technologies). NGS libraries were prepared in 96 well format using NexteraXT DNA Sample Preparation Kit and NexteraXT Index Kit for 96 Indexes (Illumina) according to the manufacturer's protocol. The resulting sequencing libraries were quantified in a qPCR-based approach using the KAPA SYBR FAST qPCR MasterMix Kit (Peqlab) on a ViiA 7 real time PCR system (Life Technologies). 96 samples were pooled per lane for paired-end sequencing (2×100 bp) on Illumina Hiseq2000 or Hiseq2500 sequencers using TruSeq PE Cluster v3 and TruSeq SBS v3 sequencing chemistry (Illumina). Basic sequencing quality parameters were determined using the FastQC quality control tool for high throughput sequence data (Babraham Bioinformatics Institute).

### Data Analysis

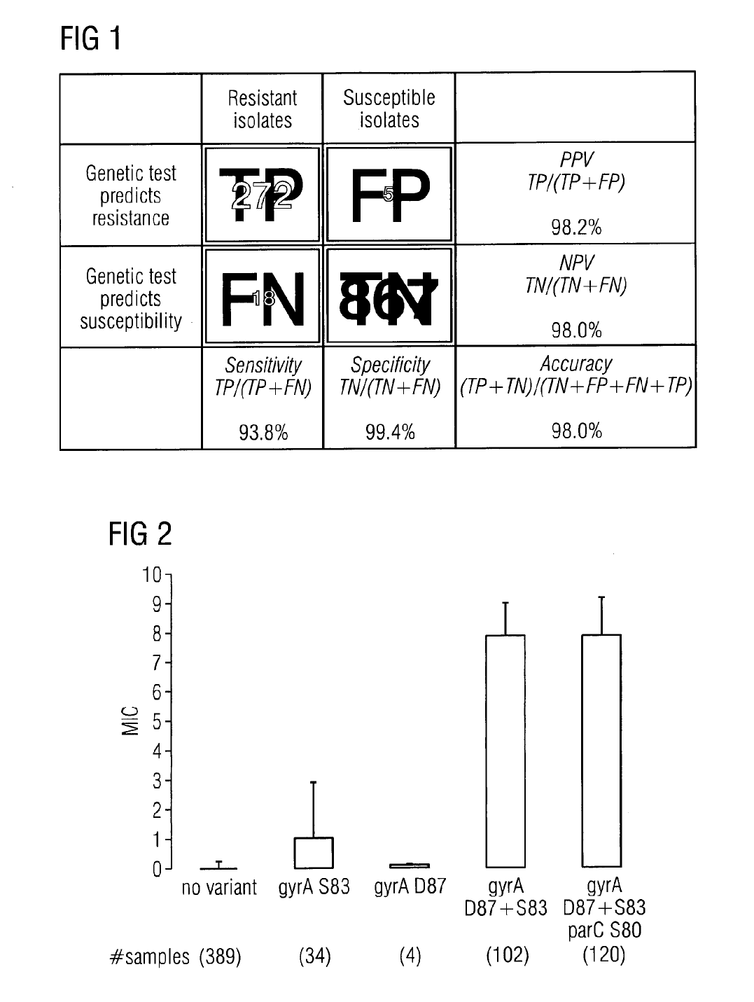

Raw paired-end sequencing data for the 1,162 E. coli samples were mapped against the E. coli DH10B reference (NC_010473)(see also above in Example 1) with BWA 0.6.1.20 The resulting SAM files were sorted, converted to BAM files, and PCR duplicates were marked using the Picard tools package 1.104 (http://picard.sourceforge.net/). The Genome Analysis Toolkit 3.1.1 (GATK)21 was used to call SNPs and indels for blocks of 200 E. coli samples (parameters: -ploidy 1-glm BOTH-stand_call_conf 30-stand_emit_conf 10). VCF files were combined into a single file and quality filtering for SNPs was carried out (QD<2.0∥FS>60.0∥MQ<40.0) and indels (QD<2.0∥FS>200.0). Detected variants were annotated with SnpEff22 to predict coding effects. For each annotated position, genotypes of all E. coli samples were considered. E. coli samples were split into two groups, low resistance group (having lower MIC concentration for the considered drug), and high resistance group (having higher MIC concentrations) with respect to a certain MIC concentration (breakpoint). To find the best breakpoint all thresholds were evaluated and p-values were computed with Fisher's exact test relying on a 2×2 contingency table (number of E. coli samples having the reference or variant genotype vs. number of samples belonging to the low and high resistance group). The best computed breakpoint was the threshold yielding the lowest p-value for a certain genomic position and drug. For further analyses positions with non-synonymous alterations and p-value <10-9 were considered. Based on the contingency table, the accuracy (ACC), sensitivity (SENS), specificity (SPEC), and the positive/negative predictive values (PPV/NPV) were calculated (FIG. 1).

Since a potential reason for drug resistance is gene duplication, gene dose dependency was evaluated. For each sample the genomic coverage for each position was determined using BED Tools. 23 Gene ranges were extracted from the reference assembly NC_010473.gff and the normalized median coverage per gene was calculated. To compare low- and high-resistance isolates the best area under the curve (AUC) value was computed. Groups of at least 20% of all samples having a median coverage larger than zero for that gene and containing more than 15 samples per group were considered in order to exclude artifacts and cases with AUC>0.75 were further evaluated.

Results

The aim of our study was to demonstrate the feasibility of genetic antimicrobial susceptibility tests (GAST), to verify our method for known resistance mechanisms, and to discover novel mechanisms. Culture-based AST were performed for 1,162 E. coli isolates and 21 antimicrobial drugs belonging to 5 different drug classes: â-lactams, fluoroquinolones, aminoglycosides, tetracyclines, and folate synthesis inhibitors. The complete list of drugs is shown in Table 1. For the same 1,162 E. coli isolates, whole genome sequencing using Illumina's HiSeq2500 instrument was carried out.

Most Significant Sites in the E. coli Genome

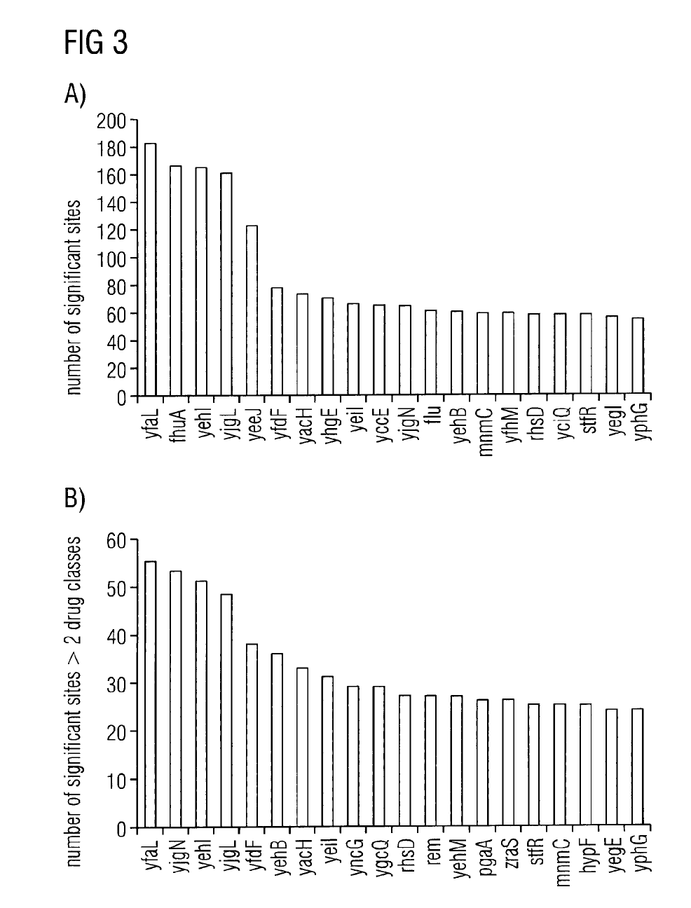

In order to calculate genome-wide significance scores, all 1,162 E. coli genomes to the reference strain DH10B were mapped. For each genomic position, the base for each sample was determined and 973,226 sites were discovered that passed the quality filtering and in which at least one sample had a non-reference base. The respective sites were correlated to the AST data for the 21 drugs using Fisher's exact test. Our analysis revealed 25,744 sites where a genetic mutation significantly correlated with at least one drug (p-value<10⁻⁹) and led to a change in the AA sequence, including point mutation and small insertions and deletions. The highest significance was reached for AA exchange S83L in GyrA and the drug Ciprofloxacin (p=10′⁵). Remarkably, GyrA is one of the targets of Ciprofloxacin. For this position, three AA exchanges, S83L, S83W, S83A, are annotated in UniProt as conferring resistance to quinolones. For this site, only 5 false positive (0.4%) and 18 false negative samples (1.6%) were discovered while 1,139 samples were identified correctly, corresponding to accuracy, specificity, and sensitivity of 98.0%, 99.4% and 93.8%, respectively (FIG. 1). Similarly, the second most significant site in GyrA, D87N/D87Y revealed just 12 false positives and 10 false negatives, the respective p-value was 10′⁶ and the accuracy 98.1%. Again, for this site the D87N exchange is annotated as conferring quinolone resistance in UniProt. For the third and fourth most significant sites, located in the second Ciprofloxacin target, ParC, (S80I, E84G), resistance related variants have also been described. In FIG. 2, the means and standard deviations of MICs for Ciprofloxacin are presented for samples having no variant in GyrA (583/D87) and ParC (S80), samples having only one mutation either in GyrA S83 or D87 and not ParC, samples having both mutations in GyrA and not ParC, and samples having all three mutations. Interestingly, the mean MIC values increase from below 1.0 for no or single mutants to above 7.8 for double or triple mutants, which shows that a combination of mutations is necessary to reach a higher level of resistance against Ciprofloxacin in this case.

Besides the mutations in type II topoisomerase drug targets (GyrA/ParC), mutations in genes ygiF (A110T, p=10⁻⁶⁷, acc=86%, spec=89.5%, sens=69.9%) and ygjM (A68V, p=10⁻⁶³, acc=89.9%, spec=94.4%, sens=67.1%) have also a high significance. Compared to the above-described AA exchanges, these two sites demonstrate a substantially decreased sensitivity and positive predictive values (PPV). While the PPV for the four AA exchanges in GyrA and ParC was between 94.8% and 98.2%, the PPV of these two exchanges decreases to 59.0% and 70.8%. This means that the likelihood to be resistant given the exchanged AA is almost as high as the likelihood to be susceptible given the exchanged AA, limiting the probability that the respective AA exchanges are causative.

To discover other AA exchanges that are potentially causative for drug resistance, the list of all 25,744 sites were filtered (at least 150 resistant E. coli isolates carry the AA exchange, NPV>50%, PPV>75%). This filtering revealed 127 candidate sites (see also Table 4). Besides the already described exchanges in GyrA and ParC, AA exchanges in YdjO associated with predicted resistance to different â-lactams (V121E, 5120C, V118F, 1114V, K111E, and D112N) were discovered. Likewise, AA exchanges in YcbS (E848Q, E848*), RhsC (R717Q, W492C), YcbQ (T86I), YagR (S274T) and YeaU (N293K) were reported for lactams. Finally, AA exchanges related to quinolones, tetracycline, and lactams in YhaL were discovered (altogether 23 different sites).

In addition, the most significant non-synonymous AA exchange for each drug were computed (p-value threshold<10⁻⁹). Of 21 tested drugs, only two (Imipenem, Meropenem) were not found to be associated with an AA exchange with such a low p-value. Interestingly, the S83L mutation in GyrA is the predominant exchange in 15 drugs. For the drugs Ciprofloxacin and Levofloxacin, of which GyrA is a target, the p-values were however much lower than the p-values for this mutation in association with the remaining 13 drugs (>10⁻⁶² vs.<10⁻²⁰⁹). In addition, a significant decrease in sensitivity and/or PPV in these cases were observed: either the sensitivity or PPV is below 55% for drugs, of which GyrA is not the target, demonstrating that these measures are effective for separating mutations in true targets from others.

### Mutations in Known Drug Targets

In 9 cases, mutations associated with drugs were detected in genes that are also encoding the targets for the respective drugs. This includes the mutations associated with Ciprofloxacin and Levofloxacin in GyrA (S83L, D87N, D87Y, D678E, E574D) and ParC (S80I, E84G, E84V, E84A, A192V, Q481H, A471G, T718A, Q198H), mutations associated with Cephalotin in AmpC (K40R, 1300V, T3351, A210P, Q196H, A236T, R248C), with Sulfamethoxazole in FolC (A319T, R88C, G217S), with Cefazolin in MrcB (D839E, QQQP815Q, R556C) and PbpC (L357V, V348A, A15T, A217V, Q495L, V768F, A701E, K766R, K766T, T764S, T764A, R602L, E446G, R669H, A202T) and with Ceftazidime in PbpG (A28V).

### Most Affected Genes and Multi-Drug Resistant Sites

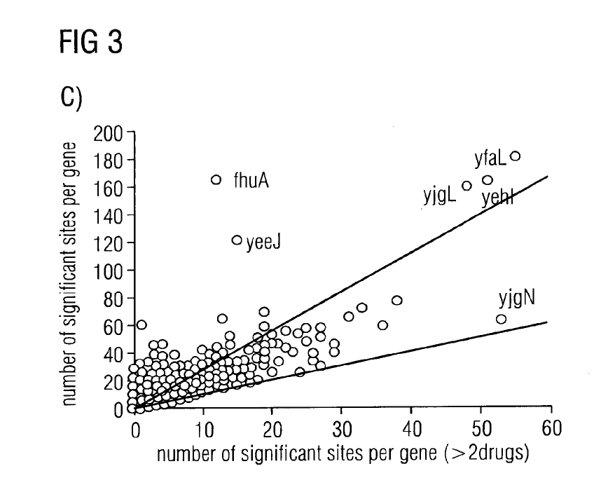

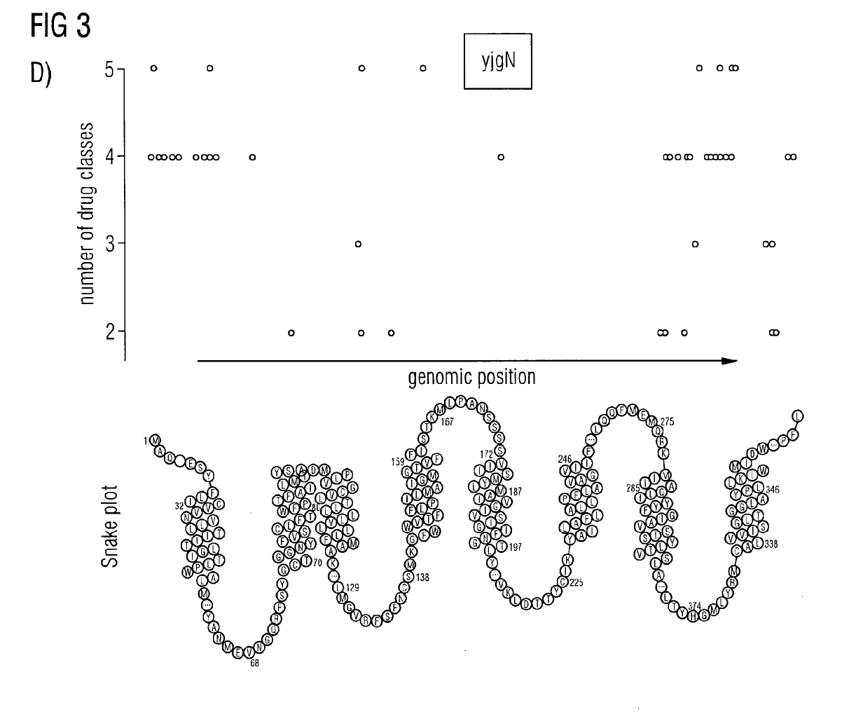

Mutations are not uniformly distributed across E. coli genes: for example, yfaL, fhuA, yehI, yjgL, and yeeJ carry over 120 non-synonymous variants per gene (FIG. 3A); in yfaL, as many as 182 significant exchanges were discovered. In order to discover sites that are relevant for multi-drug resistance, the number of AA exchanges significant in association with at least 3 drug classes were calculated (FIG. 3B) and the respective site counts for each gene plotted in FIG. 3C. On average, 35% of all significant sites were associated with at least three drugs. While three genes, yfaL, yehI, and yjgL, had the highest number of AA exchanges, yjgN had a substantially increased number of sites associated with multi-drug resistance (53 of 64 sites, 83%), while yeeJ (15 of 122 sites, 12%) and fhuA (12 of 166 sites, 7%) carry fewer sites relevant for multiple drug classes than expected. In yjgN, the positions significantly associated with multiple drug classes were concentrated in the terminal regions of the gene (FIG. 3D).

### Coverage Analysis

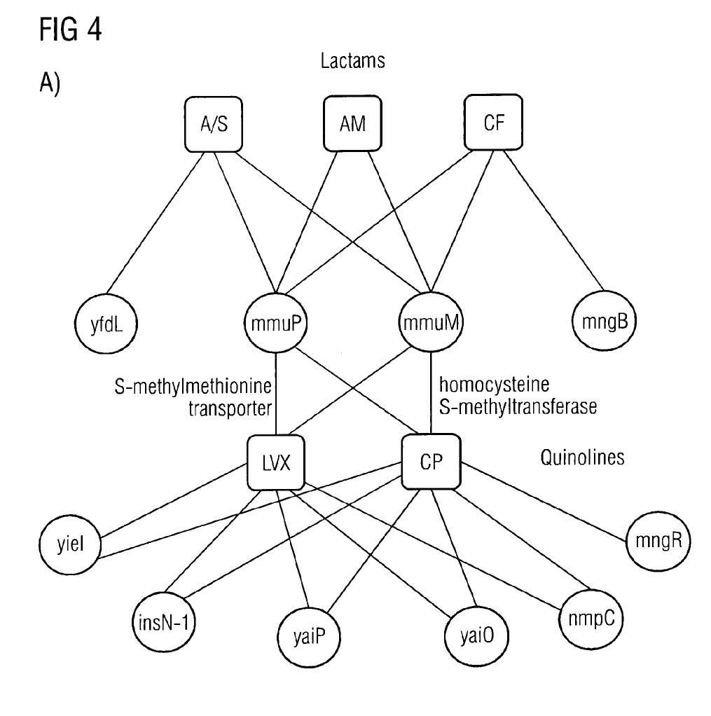

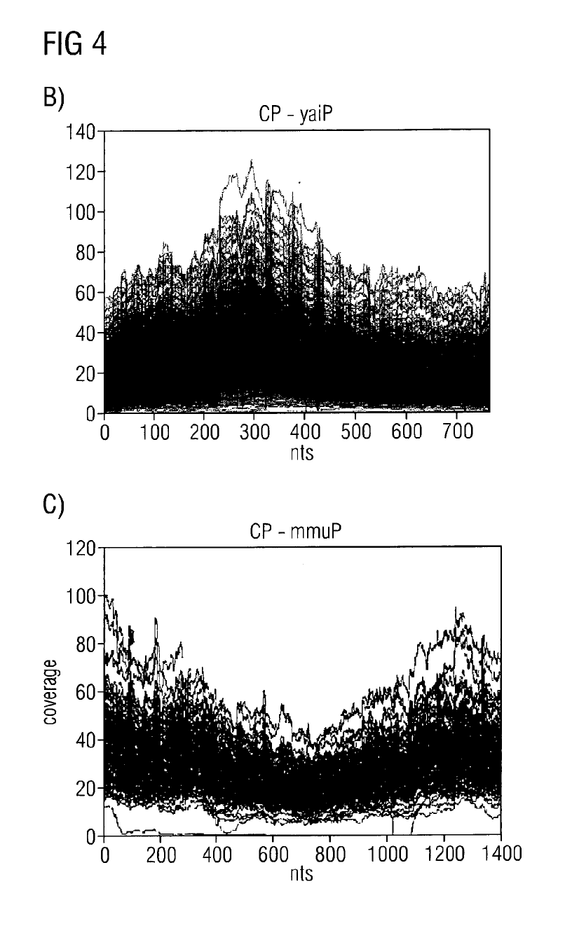

A potential reason for drug resistance is gene duplication or deletion, which may be observed in our dataset by inspecting the read coverage of different genes in the groups of resistant and susceptible isolates. To estimate the difference in coverage, AUC values were calculated for the normalized median coverage per gene in the two groups. Altogether 23 cases of abnormal differences in gene coverage between resistant and susceptible bacteria were discovered, resulting in an AUC>0.75 (FIG. 4A). Connections for three d-lactams and two quinolones are reported. Central genes are mmuP and mmuM, encoding for a putative S-methylmethionine transporter and a homocysteine S-methyltransferase, respectively, for which the coverage is substantially higher in bacteria resistant to all 5 drugs. In strains resistant to Levofloxacin and Ciprofloxacin, the inner membrane protein YieI and InsN-1, a regulator of insertion element, were likewise higher abundant. In contrast, genes encoding glucosyltransferases YaiP, YaiO, outer membrane protein NmpC and DNA-binding transcriptional repressor MngR were less covered in strains resistant to these drugs. FIGS. 4B and 4C show an example coverage plot for the lower abundant covered yaiP and the higher abundant covered mmuP in strains resistant to Ciprofloxacin. Best diagnostic accuracy was reached for Ciprofloxacin and the gene mmuP, with an AUC value of 0.923, demonstrating that this quantitative information allows for accurate separation between resistant and susceptible strains.

### DISCUSSION

The considerable and ongoing increase of infections caused by multi-drug resistant pathogens presents a major threat for patients especially in hospital settings. The development of new drugs is a long and expensive venture, and stagnated in the last years despite increasing investments in research and development. The announcement by the FDA in September 2012 to form an internal task force for supporting the development of new antimicrobial drugs emphasizes the importance of this topic. Until these drugs become available, it has to be learned how to apply the available ones most efficiently. Abundant prescribing of broad-spectrum antibiotics promotes the development of multi-drug resistance, so a more careful selection of drugs is needed. Thus, methods that may quickly stratify patients and provide them with the optimal therapy are needed. Identifying the genetic loci in the infectious agent that are predominantly responsible for an observed resistance or susceptibility is a crucial point for this.

Here, 1,162 clinical isolates of E. coli were analyzed for their susceptibility towards 21 FDA approved drugs and combined this information with whole genome NGS data to identify potential variants that might be causative for the observed resistance patterns. In total, 25,744 significant sites were found (p-value <10⁻⁹). The method correctly identified already known drug targets in nine gene/drug combinations: gyrA (Ciprofloxacin, Levofloxacin), parC (Ciprofloxacin, Levofloxacin), ampC (Cephalothin), folC (Trimethoprim Sulfamethoxaxole), mrcB (Cefazolin), pbpC (Cefazolin), and pbpG (Ceftazidime). To identify other potential sites that might be secondary drug targets, filtering criteria were applied using the measures NPV/PPV, which provided a reduction in the number of potentially relevant sites from 25,744 to 127 sites.

Considering the best drug-target combinations according to the computed p-values, the AA exchange S83L in GyrA was found to be the predominant mutation for 15 drugs. Since only Ciprofloxacin or Levofloxacin are approved drugs for GyrA, the other associations to this protein might be a side-effect of multi-drug resistance. Employing additional measures such as sensitivity, PPV, and NPV facilitates the separation of causative drug targets from other variants as exemplified in this case.

Instead of using only single variants, a combination of several variant positions may improve the prediction accuracy and further reduce false positive findings that are influenced by other factors.

Since gene duplication and/or deletion might also play a role in resistance development mechanisms, the gene coverage combined with the resistance data was analyzed and 23 cases of abnormal differences in gene coverage between resistant and susceptible bacteria were discovered. Interestingly, an increase of genetic material in resistant bacteria, (e.g. for genes mmuP, mmuM, and yieI), was found, but also a decrease in certain genes such as mngB and mngR was found. While for membrane or transporter proteins both an increase or a decrease of gene dosage may influence drug susceptibility by not allowing a drug to permeate the membranes or to more efficiently transport it out of the cell, a decrease of the quantity of metabolic enzymes or transcription factors is not as easily interpretable in this context, and might be more or less directly related to the fitness of the isolates.

Another source of information that might improve the accuracy of our analysis are the strain-specific plasmids. Mapping the sequencing data against those plasmids will extend our knowledge about additional resistance mechanisms. In a first approach, a subset of sequencing data to about 300 E. coli plasmids was mapped. Among the genes having the most significant variant sites were e.g. repA1, trbI, psiB, and traG that are directly involved in replication, plasmid transfer, and maintenance and might play an indirect role in resistance development by giving its host the ability to facilitate spreading of resistance genes.

Compared to approaches using MALDI-TOF MS, the present approach has the advantage that it covers almost the complete genome and thus enables us to identify the potential genomic sites that might be related to resistance. While MALDI-TOF MS may also be used to identify point mutations in bacterial proteins³³, this technology only detects a subset of proteins and of these not all are equally well covered. In addition, the identification and differentiation of certain related strains may not be feasible.

The present method allows to compute a best breakpoint for the separation of isolates into resistant and susceptible groups. A flexible software tool was designed that allows to consider besides the best breakpoints also values defined by different guidelines (e.g. European and US guidelines), preparing for an application of the GAST in different countries.

Another critical point of this study is that it analysis only included cultured bacteria strains. Several studies used culture-independent samples from urine, fecal samples, or vaginal swab and applied NGS to identify or characterize the pathogens directly. The advance of the NGS technology, including the development of new long read sequencers as PacBio and Oxford Nanopore, will further improve and speed up our procedure in the future to develop a culture-independent diagnostic test based on NGS data.

This approach is capable of identifying mutations in genes that are already known as drug targets, as well as detecting potential new target sites.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method of determining an antibiotic resistance profile for a bacterial microorganism belonging to the species E. coli comprising the steps of
a) providing a sample containing or suspected of containing the bacterial microorganism;
b) determining the presence of a mutation in at least one gene of the bacterial microorganism selected from the group of genes listed in Table 4;
wherein the presence of a mutation is indicative of a resistance to an antibiotic drug.

2. The method of claim 1, wherein step b) comprises determining the presence of a mutation in at least two or more genes selected from the group of Table 4, and wherein the presence of a mutation in at least two genes is indicative of a resistance to an antibiotic drug.

3. The method of claim 1, wherein step b) comprises determining the presence of a mutation in at least one gene selected from the group of genes listed in Table 5, and wherein the presence of a mutation in said at least one gene is indicative of a resistance to an antibiotic drug.

4. The method of claim 3, wherein the presence of a mutation in at least one gene selected from the group of hofB, ymdC, potB, ycgK, ycgB, and yjjJ is determined.

5. The method of one or more of the preceding claims, where the method involves determining the resistance of E. coli to one or more antibiotic drugs.

6. The method of one or more of the preceding claims, wherein the antibiotic drug is selected from lactam antibiotics and the presence of a mutation in the following genes is determined: chbG, eutQ, flgL, gudD, gyrA, ldrA, menE, murB, murP, nepI, parC, pphB, ptrB, rhaD, ydiU, yegE, yegI, yfbL, yfiK, ygcR, ygiF, ygjM, yohG, and/or yrfB.

7. The method of one or more of claims 1-5, wherein the antibiotic drug is selected from quinolone or aminoglycoside antibiotics and the presence of a mutation in the following genes is determined: agaD, chbG, eutE, eutQ, gcvP, gspO, gyrA, livG, menE, nepI, parC, speC, tiaE, torZ, uidB, yegE, yegI, yejA, ygcU, ygfZ, ygiF, ygjM, yjjU, yjjW, ymdC, ypdB, yqjA, and/or ytfG.

8. The method of one or more of claims 1-5, wherein the antibiotic drug is selected from tetracycline antibiotics and the presence of a mutation in the following genes is determined: astE, chbG, eutQ, flgL, gudD, gyrA, hemF, hypF, kdpE, ldrA, menE, murB, murP, nepI, ompC, parC, pphB, ptrB, and/or rhaD.

9. The method of one or more of claims 1-5, wherein the antibiotic drug is selected from trimethoprim sulfmethoxazol and the presence of a mutation in the following genes is determined: astE, chbG, eutQ, flgL, gudD, gyrA, ldrA, menE, murB, nepI, parC, ycjX, ydiU, yegE, yfiK, ygcR, ygiF, and/or yrfB.

10. The method of one or more of the preceding claims, wherein determining the nucleic acid sequence information or the presence of a mutation comprises determining the presence of a single nucleotide at a single position in a gene.

11. The method of one or more of the preceding claims, wherein the presence of a single nucleotide polymorphism or mutation at a single nucleotide position is detected.

12. The method of one or more of the preceding claims, wherein the mutation is a mutation which is selected from the group of mutations listed in Table 2 and/or Table 7.

13. The method of one or more of the preceding claims 1-11, wherein the presence of a mutation in at least one gene selected from the group of Table 6 is determined.

14. The method of one or more of the preceding claims, wherein the antibiotic drug is selected from the group consisting of ampicillin sulbactam (A.S.), ampicillin (AM), amoxicillin clavulanate (AUG), aztreonam (AZT), ceftriaxone (CAX), ceftazidime (CAZ), cefotaxime (CFT), cefepime (CPM), ciprofloxacin (CP), ertapenem (ETP), levofloxacin (LVX), cefuroxime (CRM), piperazillin tazobactam (P/T), trimethoprim sulfamethoxazole (T/S), tobramycin (TO), gentamicin (GM), cefazolin (CFZ), cephalotin (CF), imipenem (IMP), meropenem MER) and tetracycline (TE).

15. The method of claims 1-14, wherein the antibiotic drug is AM and a mutation in at least one of the following nucleotide positions is detected: 2428183, 4525576, 1684413, 4636902, 1181357, 206427.

16. The method of claims 1-14, wherein the antibiotic drug is A/S and a mutation in at least one of the following nucleotide positions is detected: 2428183, 4054212, 1974644.

17. The method of claims 1-14, wherein the antibiotic drug is AUG and a mutation in the following nucleotide position is detected: 2463877.

18. The method of claims 1-14, wherein the antibiotic drug is AZT and a mutation in at least one of the following nucleotide positions is detected: 2428183, 1615473.

19. The method of claims 1-14, wherein the antibiotic drug is CAX and a mutation in at least one of the following nucleotide positions is detected: 2428183, 1615473.

20. The method of claims 1-14, wherein the antibiotic drug is CFT, CP, CPE, CRM, GM, LVX, TO, T/S or CAZ and a mutation in the following nucleotide position is detected: 2428183.

21. The method of claims 1-14, wherein the antibiotic drug is ETP and a mutation in at least one of the following nucleotide positions is detected: 2052365, 2233638, 4553471, 2565236.

22. The method of claims 1-14, wherein the antibiotic drug is P/T and a mutation in at least one of the following nucleotide positions is detected: 2233638, 2216164, 2725302, 1567286, 2755319, 319290, 3240296, 1517573, 2178525, 2924554, 1516808, 37032, 1368519, 4575887.

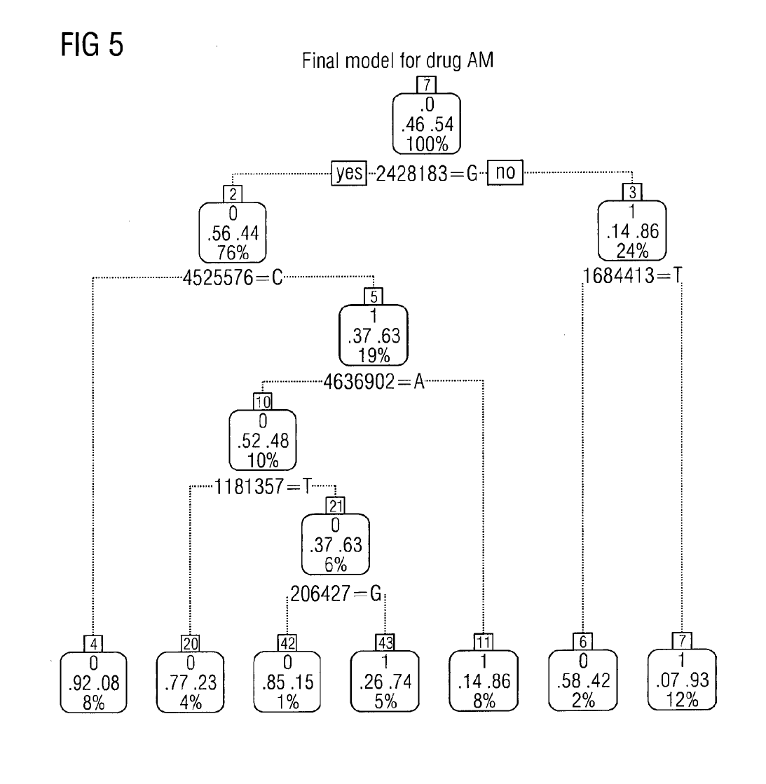

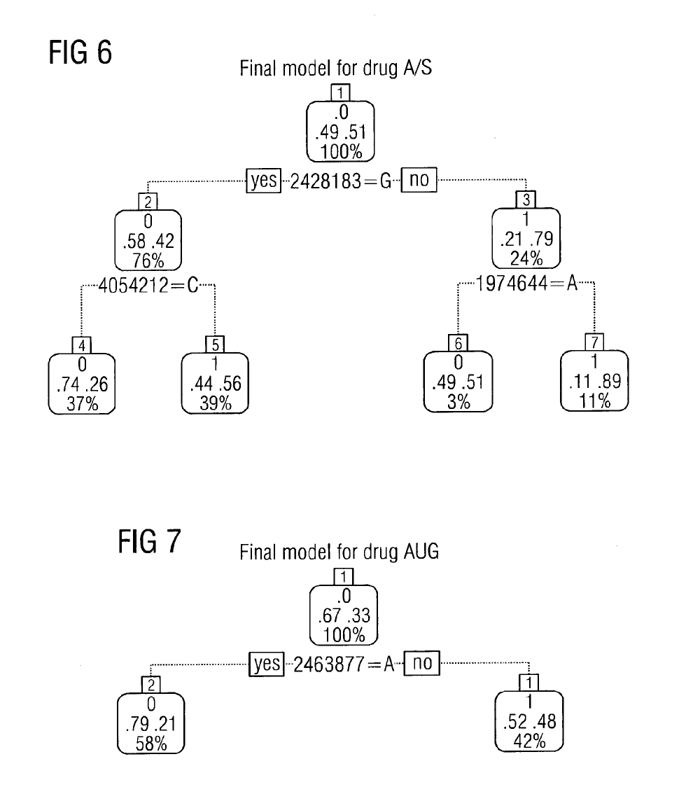

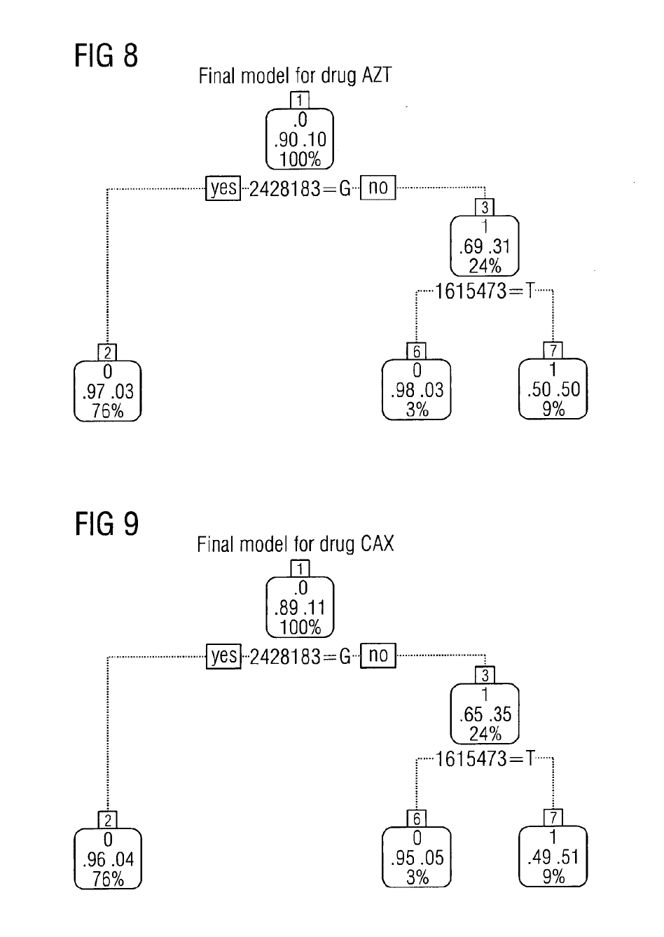

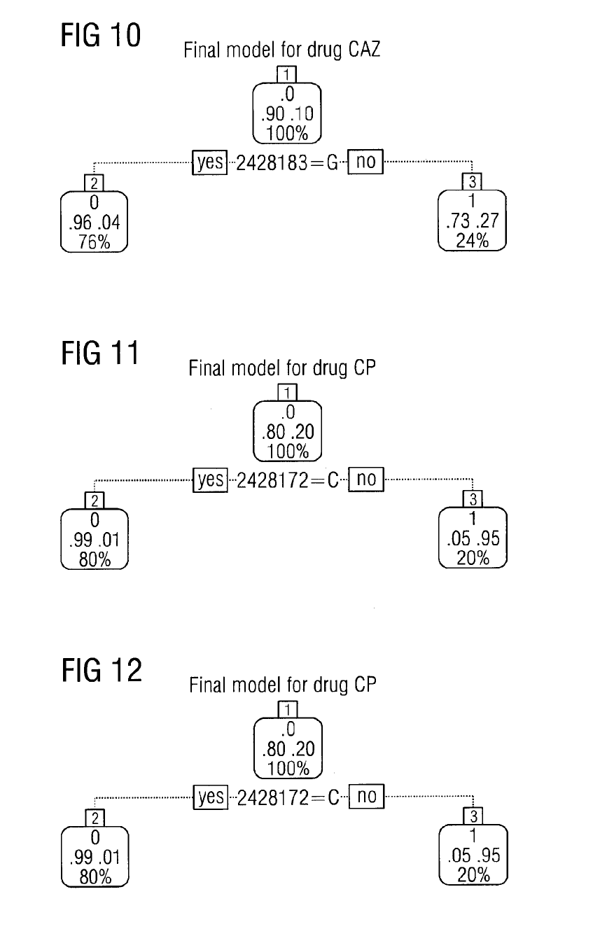

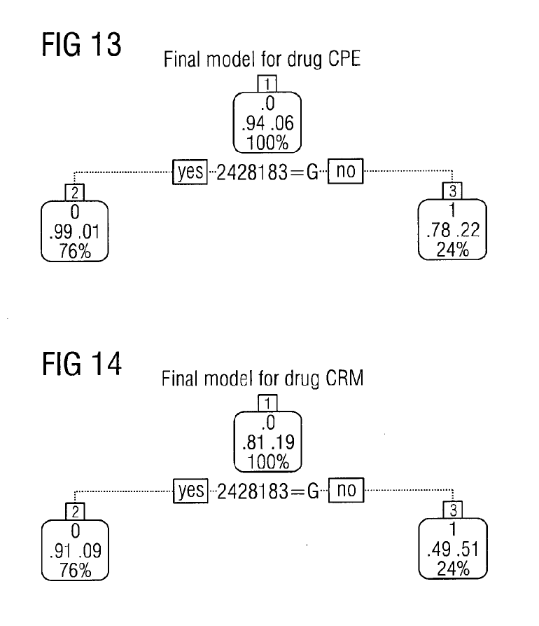

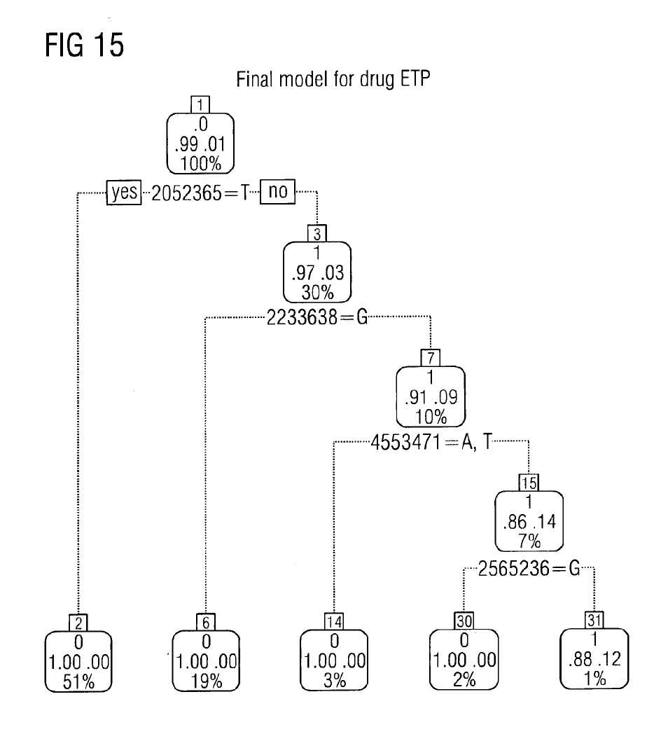

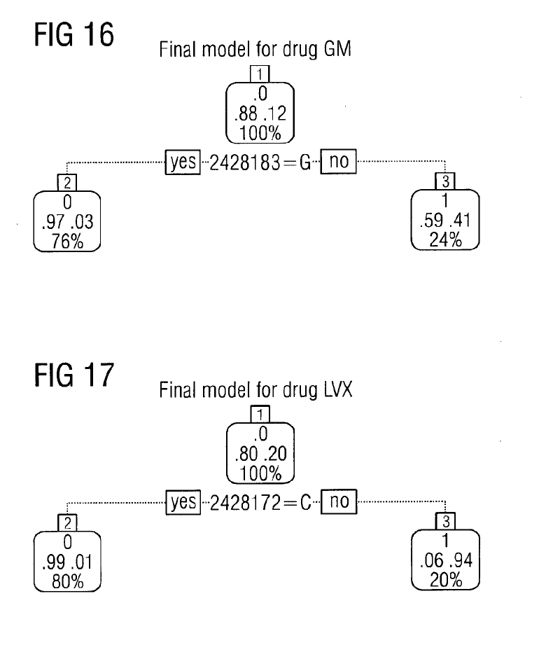

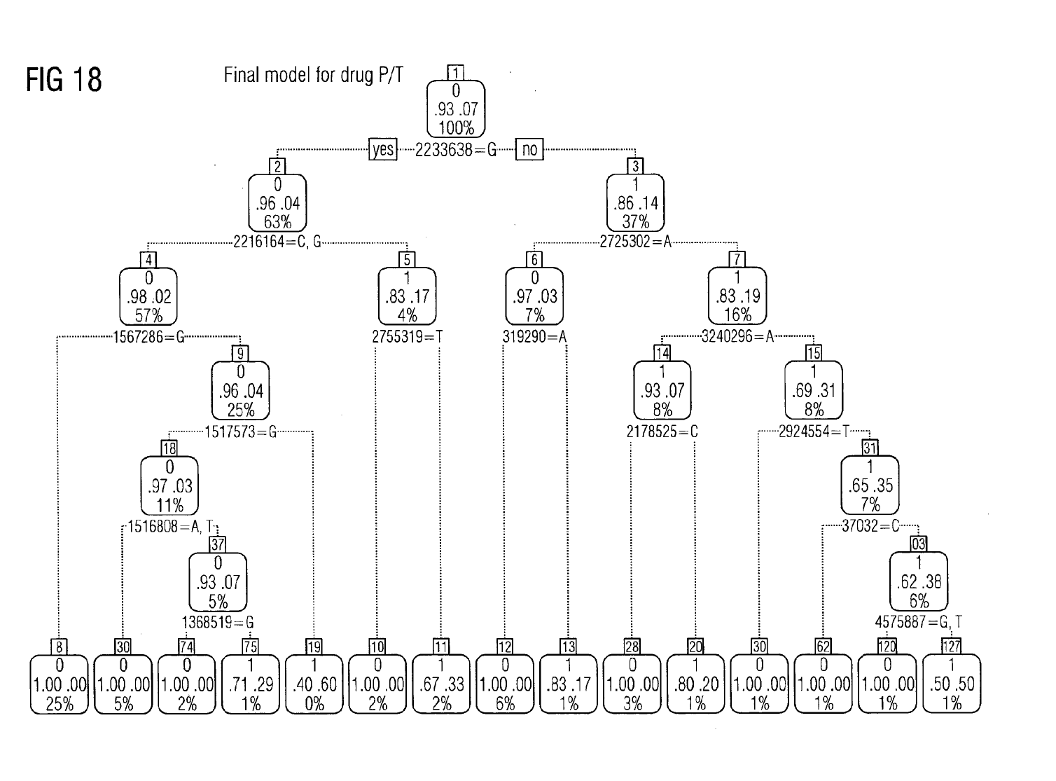

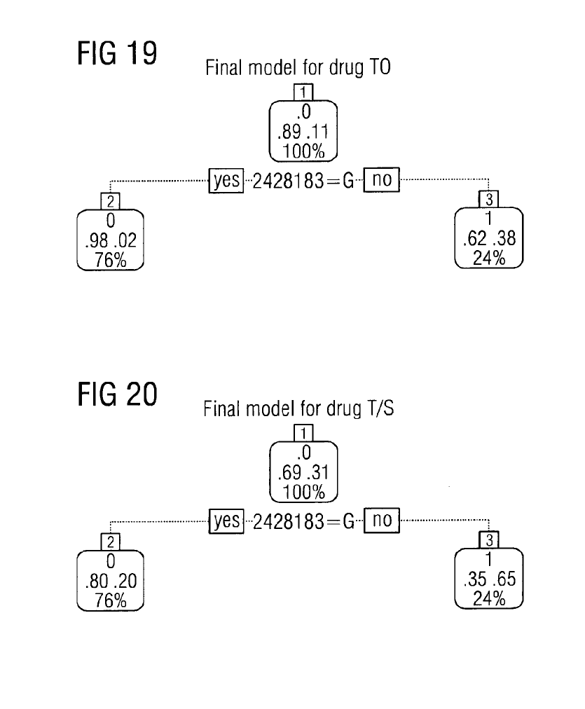

23. The method of one or more of the preceding claims 15-22, wherein the resistance to the respective antibiotic drug is tested according to the decision diagram of FIGS. 5-20.

24. The method of one or more of the preceding claims 14-22, wherein the resistance of a bacterial microorganism belonging to the species E. coli against 1, 2, 3, 4, 5, 6, 7, 8, 9, 10, 11, 12, 13, 14, 15 or 16 antibiotic drugs is determined.

25. The method of one or more of the preceding claims, wherein determining the nucleic acid sequence information or the presence of a mutation comprises determining a partial sequence or an entire sequence of the at least one gene.

26. The method of one or more of the preceding claims, wherein determining the nucleic acid sequence information or the presence of a mutation comprises determining a partial or entire sequence of the genome of said bacterial microorganism, wherein said partial or entire sequence of the genome comprises at least a partial sequence of said at least one gene.

27. The method of one or more of the preceding claims, wherein the sample is a patient sample (clinical isolate).

28. The method of one or more of the preceding claims, wherein determining the nucleic acid sequence information or the presence of a mutation comprises using a next generation sequencing or high throughput sequencing method.

29. The method of claim 28, wherein a partial or entire genome sequence of the bacterial organism is determined by using a next generation sequencing or high throughput sequencing method.

30. The method of claim 2, wherein determining the nucleic acid sequence information or the presence of a mutation comprises determining a nucleic acid sequence information or mutation of 3, 4, 5, 6, 7, 8 or 9 genes selected from Table 4.

31. The method of claim 3, wherein determining the nucleic acid sequence information or the presence of a mutation comprises determining a nucleic acid sequence information or mutation of 2, 3, 4, 5, 6, 7, 8 or 9 genes selected from Table 5.

32. The method of claim 31, wherein the method of the invention further comprises determining the resistance to 2, 3, 4, 5, 6 or more antibiotic drugs.
